(19) United States
(12) Patent Application Publication
(10) Pub. No.: US 2019/0302097 A1
(43) Pub. Date: Oct. 3, 2019

(54) A URINE ANALYSIS SYSTEM AND A METHOD FOR URINE ANALYSIS

(71) Applicant: SHENZHEN XIAOFU MEDICAL TECHNOLOGY CO., SHENZHEN, GUANGDONG PROVINCE (CN)

(72) Inventors: ZHENXING NIU, SHENZHEN, GUANGDONG PROVINCE (CN); DA WANG, SHENZHEN, GUANGDONG PROVINCE (CN)

(73) Assignee: SHENZHEN XIAOFU MEDICAL TECHNOLOGY CO., SHENZHEN, GUANGDONG PROVINCE (CN)

(21) Appl. No.: 16/307,835

(22) PCT Filed: Aug. 1, 2016
(86) PCT No.: PCT/CN2016/092713

§ 371 Date: Dec. 6, 2018

(30) Foreign Application Priority Data
Jun. 6, 2016 (CN) 201610394118.6

(51) Int. Cl. G01N 33/493; G01N 1/14; G01N 21/78; B01L 3/00

(52) U.S. Cl. CPC G01N 33/493; G01N 1/14; G01N 21/78; B01L 2300/0832; B01L 2300/0803; B01L 2300/0825; B01L 3/5027

(57) ABSTRACT

The present invention is a urine analysis system, including a urine analyzer and a micro-fluidic chip, wherein the micro-fluidic chip is provided with several test strips and fluid channels corresponding to each test strip; and the liquid inlet end is arranged at the lower end of the micro-fluidic chip and connected to one end of the fluid channel. The liquid outlet end is arranged at the upper end of the micro-fluidic chip and connected to the other end of the fluid channel. The urine analyzer comprises a detector for detecting the test strips, a connection port for the micro-fluidic chip; and a suction device which connects the liquid outlet end of the micro-fluidic chip through the connection port. The manual operation part of this case is only to install the micro-fluidic chip into the urine analysis system, which not only avoids the time uncertainty caused by the manual sampling, but also makes the entire operation process more sanitary and safer.

### TECHNICAL FIELD

The present invention relates to medical apparatus and microfluidics, in particular to a urine analysis system and a method for urine analysis.

### BACKGROUND

Urine analysis is an important means of clinical examination. The urine analysis system is usually used in hospitals and medical institutions. The basic working mode of the urine analysis system is to calculate the concentration of chemical components in urine by using the chemical reaction between the test strips and the components in urine and irradiating the test strips in light.

Using the urine analysis system for urine analysis, we need to use test strips or test paper. The test strip is usually made into a long strip which have multiple rectangular test papers on one side, each test paper corresponds to a test item. The test paper is based on absorbent material (filter paper), and it is made of a specific reagent. The test strip is easy to absorb water, when the test strip is immersed in urine, the test strip is inhaled into the urine. Then the chemical components in the urine will react with the specific pre-added reagent, which leads to the color change in the test strip.

Because the color change of the test strip is caused by chemical reaction, while some chemical reaction, especially enzymatic reaction takes time, therefore, reaction time is directly related to color change. The urine analysis system usually has a strict setting on the reaction time of the test strips, too long or too short reaction time will make the detection results inaccurate. The urine analysis system using test strips is generally divided into semi-automatic and full-automatic. The full-automatic urine analysis system ensures consistency of reaction time due to automation of sampling, reactions, test strips delivering and color data collection. However, for the semi-automatic urine analysis system, it is necessary to immerse test strips into urine manually, and put the test strips into the test area of the semi-automatic urine analyzer manually, then the semi-automatic urine analyzer will deliver the test strips and obtain color data in the specific time. Normally, after the test strip is immersed in urine, the operator will eliminate the test strip with an absorbent paper or other absorbent device to remove excess urine from the test strip. Such a manual operation is completely impossible to define an accurate operation time for all the test strips. As a result, in terms of the semi-automatic urine analysis system, the reaction time of the test strips cannot be defined as well as the accuracy of the test results cannot be guaranteed. In addition, the optical system of the urine analysis system generally obtains color data from a front side of the test strip, the urine is also immersed in the front side of the test strip, so the manual operation of the sampling has great uncertainty, which causes urine to stay on the surface of the test strip and further change the reflectivity of the test strip surface, thus affecting the accuracy of the color data.

### SUMMARY OF THE INVENTION

The present invention aims to provide a method of infiltrating the test strips by automatic absorbing urine through chip, a urine analysis system with higher accuracy and a method for urine analysis to overcome the deficiency of existing technology,

In order to achieve the above objects, the present invention is realized through the following technical solutions.

A urine analysis system comprising a urine analyzer and a micro-fluidic chip, wherein:

the micro-fluidic chip is provided with several test strips and fluid channels corresponding to each test strip; and

the liquid inlet end arranged at a lower end of the micro-fluidic chip and connected to one end of the fluid channel,

the liquid outlet end arranged at an upper end of the micro-fluidic chip and connected to the other end of the fluid channel,

the urine analyzer comprising a detector for detecting the test strips, a connection port for the micro-fluidic chip, and

the suction device connecting the liquid outlet end of the micro-fluidic chip through the connection port.

It is preferred that the test items of the urine analysis system comprise but are not limited to one or more of urobilinogen, bilirubin, ketone, nitrite, erythrocyte, leukocyte esterase, specific gravity, PH, protein, microalbumin (MALB), glucose, ascorbic acid, creatinine, calcium, color.

It is preferred that, in the urine analysis system, N fluid channels and N test strips are arranged in the N fluid channels, each test strip corresponds to one fluid channel, and N is an integer not less than 1 and not greater than 15. The test strips react with the urine sample passing through the fluid channel and change color.

It is preferred that the urine analysis system, at least one of the two larger sides of the test strip area is encapsulated in transparency.

It is preferred that the urine analysis system, at least one of the two larger sides of the test strip area is encapsulated in exposure.

It is preferred that the urine analysis system, wherein the shape of the micro-fluidic chip is rectangle, and the fluid channels are parallelly distributed on the micro-fluidic chip.

It is preferred that the urine analysis system, wherein the shape of the micro-fluidic chip is discoid, and the fluid channels are radially distributed on the micro-fluidic chip.

It is preferred that the urine analysis system, wherein the shape of the micro-fluidic chip is cylindrical, and the test strips are distributed on the cylindrical side.

It is preferred that the urine analysis system, wherein the shape of the micro-fluidic chip is a circular truncated cone, and the test strips are distributed on the truncated cone side.

It is preferred that the urine analysis system, wherein the urine analyzer includes an unloading device for unloading the micro-fluidic chip.

It is preferred that the urine analysis system, wherein the detector is arranged by the side of the micro-fluidic chip and includes a movable detection sensor.

It is preferred that the urine analysis system, wherein the detector includes N detection sensors, and the number of detection sensors is the same as the number of test strips.

It is preferred that the urine analysis system, wherein the detector includes a detection sensor and a mechanical device that moves the micro-fluidic chip. The detection sensor is used to collect the color data of the test strips.

It is preferred that the urine analysis system, wherein the urine analyzer also includes a top shell, a side shell and a bottom shell, and the top of the urine analyzer is provided with a display screen, a sampling key and a unloading key. The unloading device is connected under the unloading key. The shape of suction device is cylindrical and the unloading device is nested outside the suction device. The suction device is connected with the connection port through an internal pipe.

It is preferred that the urine analysis system, wherein the detector is located in flank of the micro-fluidic chip, the detector also includes: a main circuit board which is fixed at a frame, a linear motor with a lead screw and two parallelly sliding guide rods are arranged inside the frame, and a mobile circuit board stretch across the two sliding guide rods which is connected with the linear motor with lead screw. The detection sensor is arranged on the mobile circuit board, the mobile circuit board connects with the main circuit board through flexible flat cable.

It is preferred that the urine analysis system, wherein the urine analyzer is in the shape of a handheld pipettor, including a top shell, a bottom shell, and the flank of the top shell is integrated with a main electrical component, which includes a circuit board, keys, a display screen, a battery and wireless connection module. The top of the urine analyzer is also provided with a sampling key connecting the suction device and an unloading key connecting the unloading device. The unloading device is housed concentric to the exterior of the suction device. The lower part of the suction device is connected with the micro-fluidic chip as the connection port.

It is preferred that the urine analysis system, wherein the detector includes a detection sensor which contains one sensor, an electrical motor used to drive the micro-fluidic chip rotating, a transmission gear, and a slewing mechanism which is used to drive the micro-fluidic chip rotating and make the test strips move to the detection sensor.

It is preferred that the urine analysis system, wherein the shell of the urine analyzer is handheld and has a display screen on the top. The shell is provided with a sampling key and an unloading key, and the shell is equipped with circuit boards and batteries inside. The urine analyzer connects the micro-fluidic chip through the connection port, and the lateral connection port is equipped with an electromagnet and an mechanical arm which is used to unload the micro-fluidic chip. The suction device and the chip unloading device are electrically controlled.

It is preferred that the urine analysis system, wherein the detector includes a rotary motor connected with the connection port, a pump connected with the connection port through a pipe, and a detection sensor located on the flank of the micro-fluidic chip.

A method for urine analysis, including the following steps:

Step 1) Loading chip. Inserting the micro-fluidic chip used for urine analysis into the urine analysis system so that the liquid outlet end of the fluid channel of the micro-fluidic chip is connected with the suction device in the urine analysis system, test strips which can react with the urine sample to be tested are placed in the micro-fluidic chip;

Step 2) Sampling. Inserting the liquid inlet end of the micro-fluidic chip into the urine sample to be tested, and starting the suction device to let the urine sample flow into the micro-fluidic chip;

Step 3) Reacting. After Step 2 and the urine sample to be tested is absorbed in the microfluidic chip, the test strips reacts with the urine sample and produce color changes;

Step 4) Obtaining color data. The urine analysis system has a detector which obtains the color data of the test strips in the micro-fluidic chip which reacts with the urine sample to be tested;

Step 5) Analyzing results. The urine analysis system analyze the concentration of components in the urine sample to obtain the urine analysis results according to the color data of the test strips obtain in the Step 4.

Step 6) Unloading the chip. Unloading the micro-fluidic chip from the urine analysis system.

It is preferred that the method for urine analysis, wherein the test items of the method for urine analysis include but are not limited to one or more of urobilinogen, bilirubin, ketone, nitrite, erythrocyte, leukocyte esterase, specific gravity, PH, protein, microalbumin(MALB), glucose, ascorbic acid, creatinine, calcium, color.

It is preferred that the method for urine analysis, N fluid channels and N test strips are arranged in the N fluid channels, each test strip corresponds to one fluid channel, and N is an integer not less than 1 and not greater than 15. The test strips react with the urine sample passing through the fluid channel and change color.

It is preferred that the method for urine analysis, wherein the shape of micro-fluidic chip is rectangle, and the fluid channels are parallelly distributed on the micro-fluidic chip.

It is preferred that the method for urine analysis, wherein the shape of micro-fluidic chip is discoid, and the fluid channels are radially distributed on the micro-fluidic chip.

It is preferred that the method for urine analysis, wherein the shape of micro-fluidic chip is cylindrical, and the test strips are distributed on the cylindrical side.

It is preferred that the method for urine analysis, wherein the shape of micro-fluidic chip is a circular truncated cone, and the test strips are distributed on the truncated cone side.

It is preferred that the method for urine analysis, wherein the urine analyzer also includes an unloading device for unloading the micro-fluidic chips.

It is preferred that the method for urine analysis, wherein the detector is arranged by the side of the micro-fluidic chip and includes a movable detection sensor. The detection sensor moves to the flank of each test strip of the micro-fluidic chip to collect the color data of the test strips.

It is preferred that the method for urine analysis, wherein the detector includes N detection sensors, and the number of detection sensors is the same as the number of test strips; the N detection sensors are respectively corresponding to the color data of the N test strips in the micro-fluidic chip.

It is preferred that the method for urine analysis, wherein the detector includes a detection sensor and a mechanical device that moves the micro-fluidic chip. The mechanical device drives the micro-fluidic chip moving so that the test strips of the microfluidic chip entering the detection sensor by order. The detection sensor is used to collect the color data of the test strips.

It is preferred that the method for urine analysis, wherein the urine analyzer also includes a top shell, a side shell and a bottom shell, and the top of the urine analyzer is provided with display screen, sampling key and unloading key. The unloading device is connected under the unloading key. The shape of suction device is cylindrical and the unloading device is nested outside the suction device. The suction device is connected with the connection port through an internal pipe.

It is preferred that the method for urine analysis, wherein the detector is located in flank of the micro-fluidic chip, the detector also includes: a main circuit board which is fixed at a frame, a linear motor with lead screw and two parallelly sliding guide rods are arranged inside the frame, and a mobile circuit board stretch across the two sliding guide rods which is connected with the linear motor with lead screw. The detection sensor is arranged on the mobile circuit board, the mobile circuit board connects with the main circuit board through flexible flat cable.

It is preferred that the method for urine analysis, wherein the urine analyzer is in the shape of a handheld pipettor, including a top shell, a bottom shell, and the flank of the top shell is integrated with a main electrical component, which includes circuit board, keys, a display screen, a battery and wireless connection module. The top of the urine analyzer is also provided with a sampling key connecting the suction device and an unloading key connecting the unloading device. The unloading device is housed concentric to the exterior of the suction device. The lower part of the suction device is connected with the micro-fluidic chip as the connection port.

It is preferred that the method for urine analysis, wherein the detector includes a detection sensor which contains one sensor, an electrical motor used to drive the micro-fluidic chip rotating, transmission gear, and a slewing mechanism which is used to drive the micro-fluidic chip rotating and make the test strips move to the detection sensor.

It is preferred that the method for urine analysis, wherein the shell of the urine analyzer is handheld and has a display screen on the top; the shell is provided with a sampling key and an unloading key, and the shell is equipped with circuit boards and batteries inside; the analyzer connect the micro-fluidic chip through the connection port, and the lateral connection port is equipped with an electromagnet and an mechanical arm which is used to unload the micro-fluidic chip; the suction device and the chip unloading device are electrically controlled.

It is preferred that the method for urine analysis, wherein the detector includes a rotary motor connected with the connection port, a pump connected with the connection port through a pipe, and a detection sensor located on the flank of the micro-fluidic chip.

It is preferred that the method for urine analysis, wherein the part of the micro-fluidic chip towards the detector is encapsulated in exposure or in transparency.

The present invention of the beneficial effects, this case of urine analysis system urges the artificial operation part of semi-automatic urine analysis system from the artificial sampling of the urine into a micro-fluidic chip installation operation, it makes the committed step of urine immersing the test strips finished automatically, and ensures the consistency of the reaction time, thus improve the detection accuracy. Without manual intervention, immersing the test strips into urine by using chip automatically leads to the higher precision and higher accuracy of semi-automatic urine analysis. The method for urine analysis in this case is also easy to operate. In the entire operation process, the only one manual operation part is to install the micro-fluidic chip into the urine analysis system, which not only avoids the time uncertainty caused by the artificial sampling, but also makes the entire operation process more sanitary and safe, which can be operated without professional training.

### BRIEF DESCRIPTION OF THE DRAWINGS

FIG. 1 illustrates a view of the urine analysis system of the present invention;

FIG. 2 illustrates a view of the micro-fluidic chip in the urine analysis system of the present invention;

FIG. 3 illustrates a view of urine sampling in the urine analysis system of the present invention;

FIG. 4 illustrates a view of collecting color data in the urine analysis system of the present invention;

FIG. 5 illustrates a view of unloading the micro-fluidic chip in the urine analysis system of the present invention;

FIG. 6 illustrates a view of the urine analysis system of the present invention;

FIG. 7 illustrates a flowchart of the method for urine analysis of the present invention;

FIG. 8 illustrates a sketch of the first better implementation of the micro-fluidic chip in the urine analysis system of the present invention;

FIG. 9 illustrates an assembly drawing of the first better implementation of the micro-fluidic chip in the urine analysis system of the present invention;

FIG. 10 illustrates a sketch of the second better implementation of the micro-fluidic chip in the urine analysis system of the present invention;

FIG. 11 illustrates an assembly drawing of the second better implementation of the micro-fluidic chip in the urine analysis system of the present invention;

FIG. 12 illustrates a sketch of the third better implementation of the micro-fluidic chip in the urine analysis system of the present invention;

FIG. 13 illustrates an assembly drawing of the third better implementation of the micro-fluidic chip in the urine analysis system of the present invention;

FIG. 14 illustrates a sketch of the fourth better implementation of the micro-fluidic chip in the urine analysis system of the present invention;

FIG. 15 illustrates an assembly drawing of the fourth better implementation of the micro-fluidic chip in the urine analysis system of the present invention;

FIG. 16a illustrates a view of the urine analyzer in the urine analysis system of the present invention;

FIG. 16b illustrates an inside construction schematic drawing of the first urine analyzer in the urine analysis system of the present invention;

FIG. 16c illustrates assembly drawing of the first urine analyzer in the urine analysis system of the present invention;

FIG. 16d illustrates a view of the first detector in the urine analysis system of the present invention;

FIG. 17a illustrates a view of the second urine analyzer in the urine analysis system of the present invention;

FIG. 17b illustrates an inside construction schematic drawing of the second urine analyzer in the urine analysis system of the present invention;

FIG. 17c illustrates a view of the second detector in the urine analysis system of the present invention;

FIG. 18a illustrates a view of the third urine analyzer in the urine analysis system of the present invention;

FIG. 18b illustrates an inside construction schematic drawing of the third urine analyzer in the urine analysis system of the present invention;

FIG. 18c illustrates assembly drawing of the third urine analyzer in the urine analysis system of the present invention;

FIG. 18d illustrates a view of the third detector in the urine analysis system of the present invention;

FIG. 19a illustrates a view of the fourth detector in the urine analysis system of the present invention;

FIG. 19b illustrates an inside construction schematic drawing of the fourth detector in the urine analysis system of the present invention;

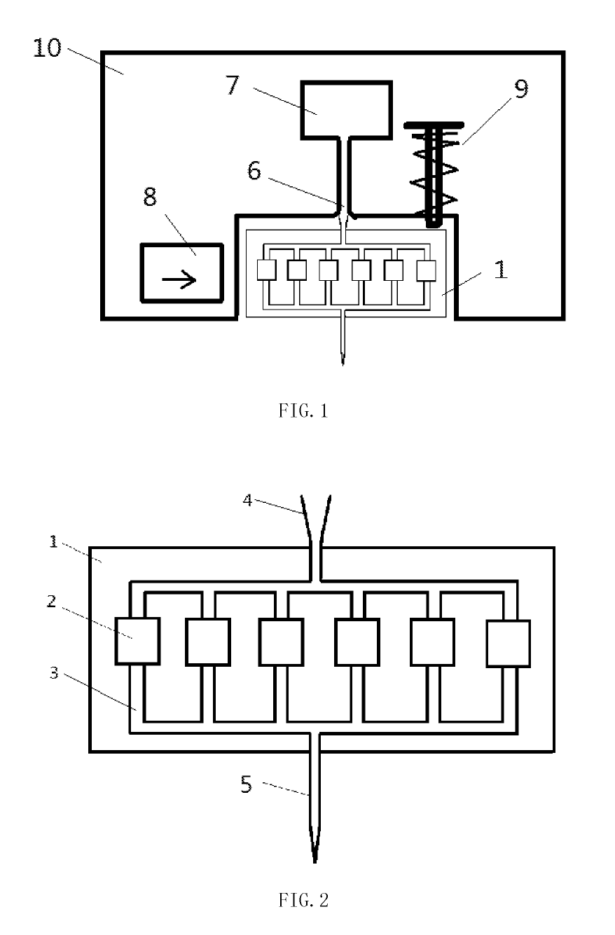

### DESCRIPTION OF THE PREFERRED EMBODIMENTS

The following further details of the present invention are introduced with the attached figures so that the technical personnel in the field can follow the instructions.

A urine analysis system, includes a urine analyzer 10 and micro-fluidic chips 1, wherein:

the micro-fluidic chip is provided with several test strips 2, and fluid channels 3 corresponding to each test strip; and

the liquid inlet end 5 is arranged at the lower end of the micro-fluidic chip and connected to one end of the fluid channel,

the liquid outlet end 4 is arranged at the upper end of the micro-fluidic chip and connected to the other end of the fluid channel,

the urine analyzer comprises a detector 8 for detecting the test strips, a connection port 6 for the micro-fluidic chip, and

the suction device 7 connects the liquid outlet end of the micro-fluidic chip through the connection port.

Furthermore, the test items of the urine analysis system include but are not limited to one or more of urobilinogen, bilirubin, ketone, nitrite, erythrocyte, leukocyte esterase, specific gravity, PH, protein, microalbumin(MALB), glucose, ascorbic acid, creatinine, calcium, color.

Furthermore, N fluid channels and N test strips are arranged in the N fluid channels, each test strip corresponds to one fluid channel, and N is an integer not less than 1 and not greater than 15. The test strip react with the urine sample passing through the fluid channel and change color.

Furthermore, at least one of two larger sides of the test strip area is encapsulated in transparency.

Furthermore, at least one of two larger sides of the test strip area is encapsulated in exposure.

A method for urine analysis, includes the following steps:

Step 1) Loading chip. Inserting the micro-fluidic chip used for urine analysis into the urine analysis system so that the liquid outlet end of the fluid channel of the micro-fluidic chip is connected with the suction device in the urine analysis system, test strips which can react with the urine sample to be tested are placed in the micro-fluidic chip;

Step 2) Sampling. Inserting the liquid inlet end of the micro-fluidic chip into the urine sample to be tested, and start the suction device to let the urine sample flow into the micro-fluidic chip;

Step 3) Reacting. After Step 2 and the urine sample to be tested is absorbed in the micro-fluidic chip, the test strips reacts with the urine sample and produce color changes.

Step 4) Obtaining color data. The urine analysis system has a detector which obtains the color data of the test strips in the micro-fluidic chip which reacts with the urine sample to be tested.

Step 5) Analyzing results. The urine analysis system analyze the concentration of components in the urine sample to obtain the urine analysis results according to the color data of the test strips obtain in the Step 4.

Step 6) Unloading the chip. Unloading the micro-fluidic chip from the urine analysis system.

Furthermore, the test items of the method for urine analysis include but are not limited to one or more of urobilinogen, bilirubin, ketone, nitrite, erythrocyte, leukocyte esterase, specific gravity, PH, protein, microalbumin(MALB), glucose, ascorbic acid, creatinine, calcium, color.

Furthermore, there are N fluid channels and N test strips are arranged in the N fluid channels, each test strip corresponds to one fluid channel, and N is an integer not less than 1 and not greater than 15. The test strips react with the urine sample passing through the fluid channel and change color.

Furthermore, the time from the test strip immersed in the urine to the detector to obtain the color data of the test strip is 30-90 seconds.

Furthermore, the part of the micro-fluidic chip towards the detector is encapsulated in exposure or in transparency.

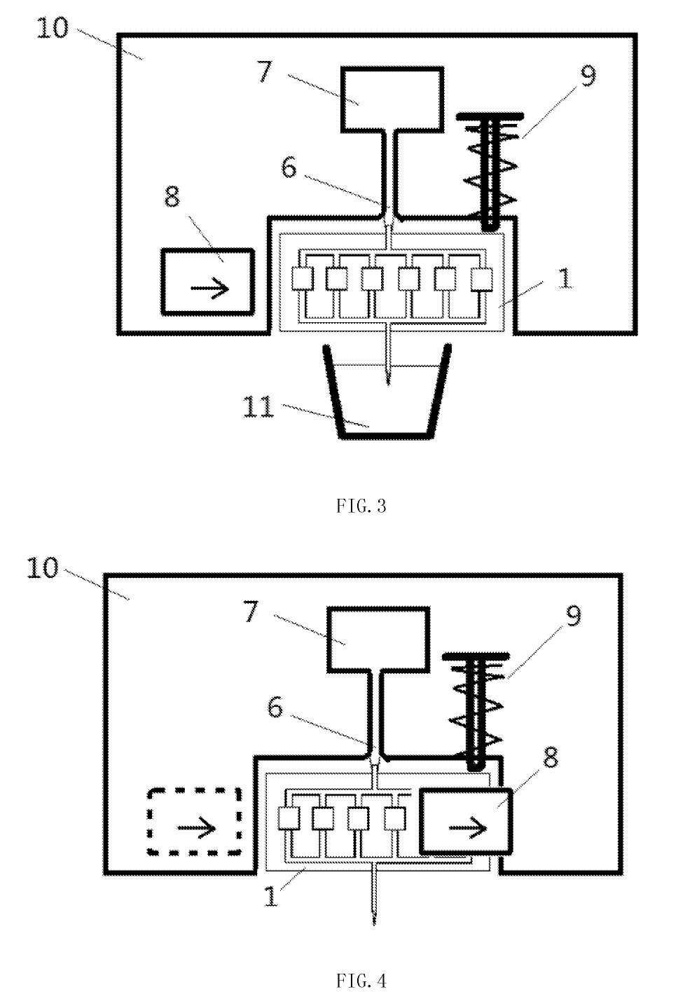

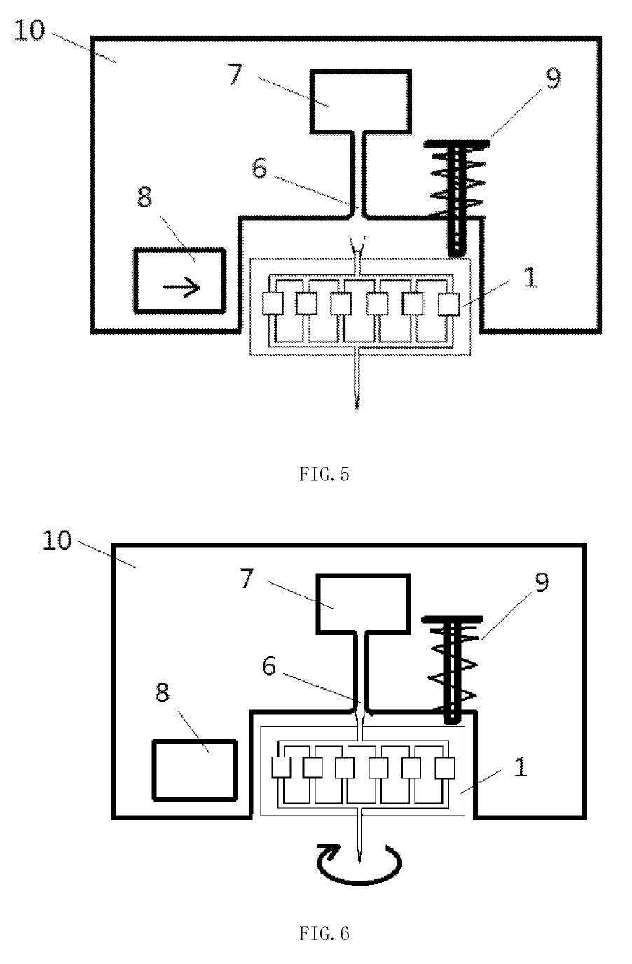

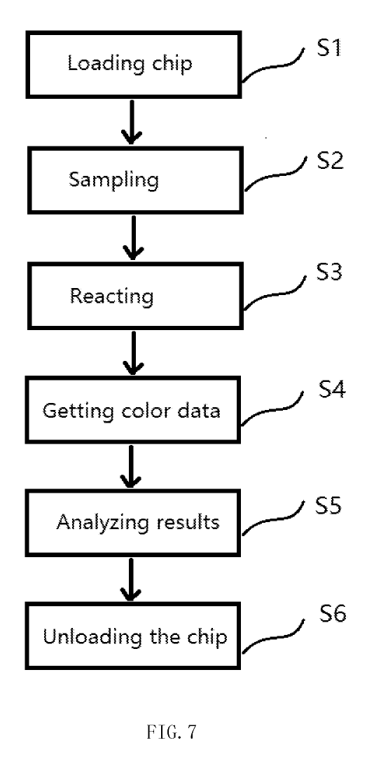

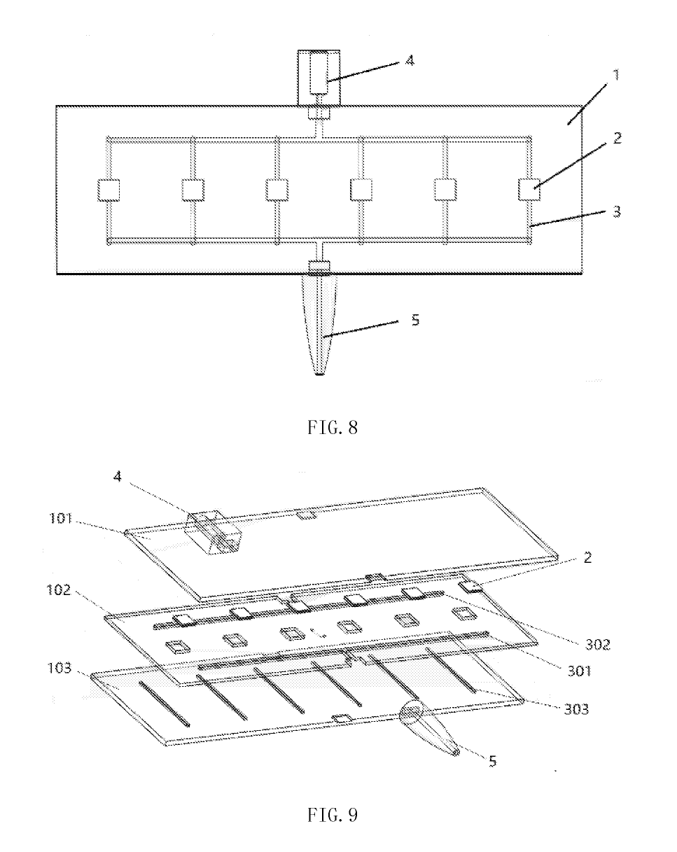

Specifically, as shown in FIG. 2, the test strip 2 was encapsulated into the micro-fluidic chip 1 which contains fluid channels 3. The shape of the fluid channels 3 ensure that the test strip 2 will be fully immersed in urine sample 11. The micro-fluidic chip 1 also contained the liquid inlet end 5 and the liquid outlet end 4. When analyzing the urine, the micro-fluidic chip 1 was inserted into the urine analyzer 10 at first to connect the suction device 7 in the urine analyzer 10 to the liquid outlet end 4 of the micro-fluidic chip 1 through the connection port 6. As shown in FIG. 3, the urine sample 11 to be tested was placed under the urine analyzer 10, so that the liquid inlet end 5 of the micro-fluidic chip 1 was immersed in the urine sample 11. The suction device 7 of the urine analyzer 10 samples, so that the urine sample 11 was sucked into the micro-fluidic chip 1 and immersed into the test strip 2. As shown in FIG. 4, after the test strip 2 was immersed in the urine sample 11 for about 30 to 90 seconds, the chemical reaction in the test strip 2 was completed. Then the detector 8 of the urine analyzer 10 moved to the micro-fluidic chip 1, and the color data of the test strip 2 in the micro-fluidic chip 1 was collected in turn. As shown in FIG. 5, the unloading device 9 in the urine analyzer 10 moved downward to separate the micro-fluidic chip 1 from the urine analyzer 10.

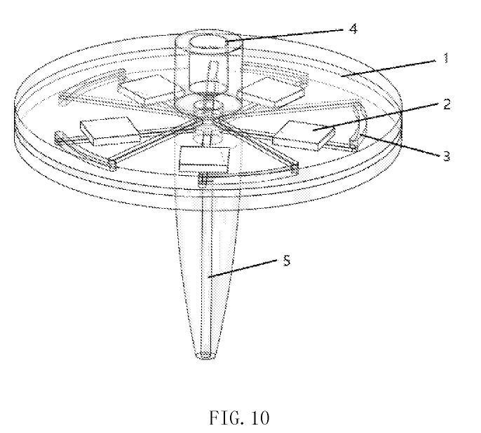

In another embodiment of the present invention, the detector 8 was fixed, while the micro-fluidic chip 1 was removable. In this embodiment, the micro-fluidic chip 1 was made into a discoid, cylindrical or truncated cone-shaped, it can undertake circular motion. The shape of the micro-fluidic chip 1 was cylindrical in FIG. 6, the detector 8 was fixed close to the test strip in the micro-fluidic chip, when we need to collect the color data of the test strip 2 in micro-fluidic chip 1, just move the micro-fluidic chip, then the detector 8 will collect all the color data of the test strips.

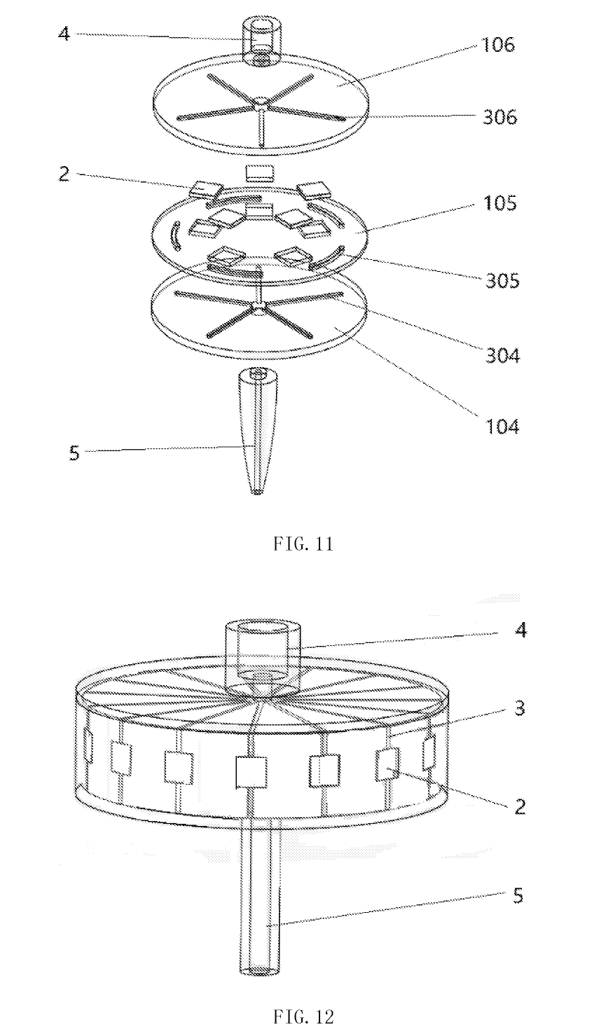

The specific operation process was shown in FIG. 2 to FIG. 7. As shown in FIG. 2, the test strip 2 was encapsulated into the micro-fluidic chip 1 which contains fluid channels 3. The shape of the fluid channels 3 ensured that the test strip 2 will be fully immersed in urine sample 11. The micro-fluidic chip 1 also contained the liquid inlet end 5 and the liquid outlet end 4. As shown in FIG. 3, when analyzing the urine, the micro-fluidic chip 1 was inserted into the urine analyzer 10 at first to connect the suction device 7 in the urine analyzer 10 to the liquid outlet end 4 of the micro-fluidic chip 1 through the connection port 6. This operation can be done manually. As shown in FIG. 4, the urine sample 11 to be tested was placed under the urine analyzer 10, so that the liquid inlet end 5 of the micro-fluidic chip 1 was immersed in the urine sample 11. The suction device 7 of the urine analyzer 10 sucked urine samples, so that the urine sample 11 was sucked into the micro-fluidic chip 1 and immersed into the test strip 2. As shown in FIG. 5, after the chemical reaction in the test strip 2 was completed, the detector 8 of the urine analyzer 10 moved to the micro-fluidic chip 1, and collected the color data of the test strip 2 in the micro-fluidic chip 1 in turn. As shown in FIG. 6, the unloading device 9 in the urine analyzer 10 moved downward to separate the micro-fluidic chip 1 from the urine analyzer 10. In the entire operation process, the only one manual operation part was to install the micro-fluidic chip into the urine analysis system, which not only avoided the time uncertainty caused by the artificial sampling, but also made the entire operation process more sanitary and safe.

As shown in FIG. 2, the micro-fluidic chip included the liquid inlet end 5, the liquid outlet end 4, and fluid channels 3 connecting the liquid inlet end 5 with the liquid outlet end 4, which is characterized in that test strips were arranged in the fluid channels, each test strip corresponded to a fluid channel, and the test strips can react with the urine sample passing through the fluid channel and change color.

The micro-fluidic chip 1 was inserted into the urine analyzer 10 to connect the suction device 7 in the urine analyzer 10 to the liquid outlet end 4 of the micro-fluidic chip 1 through the connection port 6. Then the urine sample 11 was placed to be tested under the urine analyzer 10, so that the liquid inlet end 5 of the micro-fluidic chip 1 was immersed in the urine sample 11. Next, the suction device 7 of the urine analyzer 10 sucked samples, so that the urine sample 11 was sucked into the micro-fluidic chip 1 and immersed into the test strip 2. After the chemical reaction in the test strip 2 was completed, the detector 8 of the urine analyzer 10 moved to the micro-fluidic chip 1, and collected the color data of the test strip 2 in the micro-fluidic chip 1 in turn. Finally, the unloading device 9 in the urine analyzer 10 moved downward to separate the micro-fluidic chip 1 from the urine analyzer 10.

The detector 8 of the urine analyzer 10 could be stationary, and the micro-fluidic chip 1 was removable, so that we could collect the color data of the test strip 2 in the micro-fluidic chip 1 in turn.

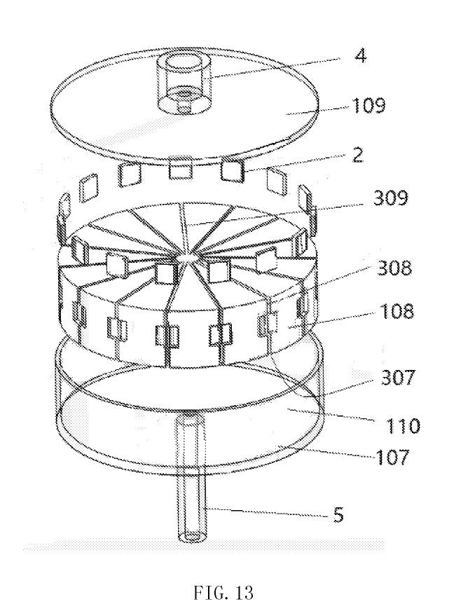

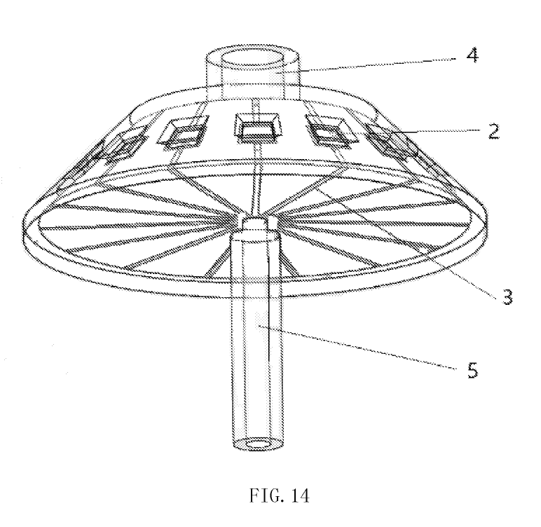

In the first better embodiment of the micro-fluidic chip in the urine analysis system, as shown in FIG. 8 (three-dimensional model diagram), the micro-fluidic chip was made into rectangle with 6 fluid channels, wherein the liquid inlet end 5 was a long, bullet-shaped catheter with internal circulation ports, wherein the liquid outlet end 4 was a square block part with larger cross-sectional circulation ports inside. As shown in FIG. 9, the micro-fluidic chip was bonded with transparent lamella 101, lamella 102 and lamella 103. The fluid channel 3 was divided into channel 301 in the transparent lamella 101, channel 302 in the lamella 102 and channel 303 in the lamella 103. The test strip was located at lamella 102. During the processing, the channel 301, the channel 302 and the test strip 2 were all connected in the thickness direction, while the channel 303 in the lamella 103 were not connected in the thickness direction. In service use, the urine sample entered micro-fluidic chip 1 from the liquid inlet end 5, it flew into the channel 303 through the channel 301 of the fluid channel 3, and then flew into channel 302, and flew out from the liquid outlet end 4. The test strip 2 was immersed in urine sample when urine sample passed through the channel 303, making the test strip 2 start chemical reaction and change color. In this embodiment, the test items of 6 test strips could be urinary protein, micro-albuminuria, glucose, ketone, PH and specific gravity. As for technician of this field, they can achieve the same goals using common means in their fields based on the transparent lamella 101, the lamella 102 and the lamella 103 of this embodiment. The common means were different forms of fluid channels, such as a simple replacement of the location of the transparent lamella 101 and lamella 103, a part of the fluid channels was put in the different lamellas, etc., these changes should be treated as equivalent as this embodiment.

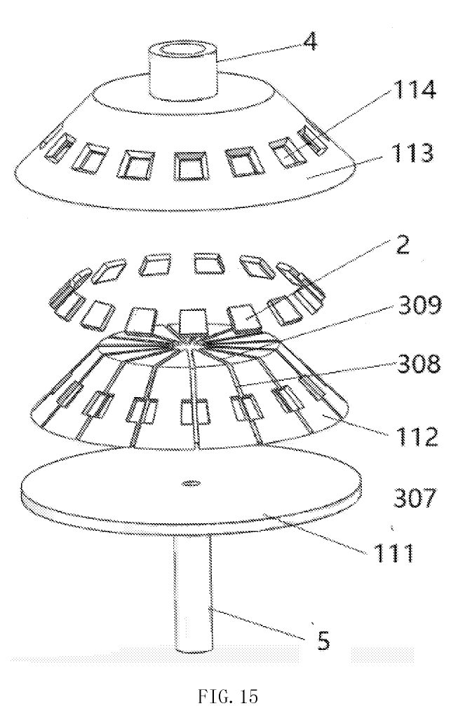

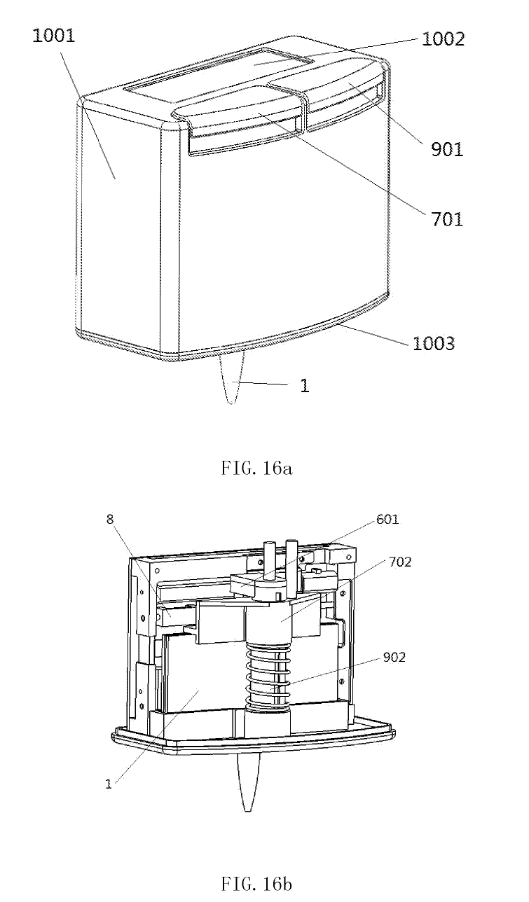

In the second better embodiment of the micro-fluidic chip in the urine analysis system, as shown in FIG. 10 (Three-dimensional model diagram), the micro-fluidic chip was made into discoid with 5 fluid channels, wherein the liquid inlet end 5 was a long, bullet-shaped catheter with internal circulation ports, wherein the liquid outlet end 4 was a cylindrical part with larger cross-sectional circulation ports inside. As shown in FIG. 11, the micro-fluidic chip was bonded with lamella 104, lamella 105 and transparent lamella 106. The fluid channel 3 was divided into channel 304 in the lamella 104, channel 305 in the lamella 105 and channel 306 in the transparent lamella 106. The test strip was located at lamella 105. During the processing, the channel 305 in the lamella 105 and the test strip 2 were all connected in the thickness direction, while the channel 304 in the lamella 104 were not connected in the thickness direction. In service use, the urine sample entered micro-fluidic chip 1 from the liquid inlet end 5, it flew into the channel 305 through the channel 304 of the fluid channel 3, and then flew into channel 306, and flew out from the liquid outlet end 4. The test strip 2 was immersed in urine sample when urine sample passed through the channel 304, making the test strip 2 start chemical reaction and change color. In this embodiment, the test items of 5 test strips could be hematuria, leukocyte esterase, nitrite, urobilinogen and bilirubin. As for technician of this field, they can achieve the same goals using common means in their fields based on the lamella 104, the lamella 105 and the transparent lamella 106 of this embodiment. The common means were different forms of fluid channels, such as a simple replacement of the location of the lamella 104 and the transparentlamella 106, put part of the fluid channels in the different lamellas, etc., these changes should be treated as equivalent as this embodiment.

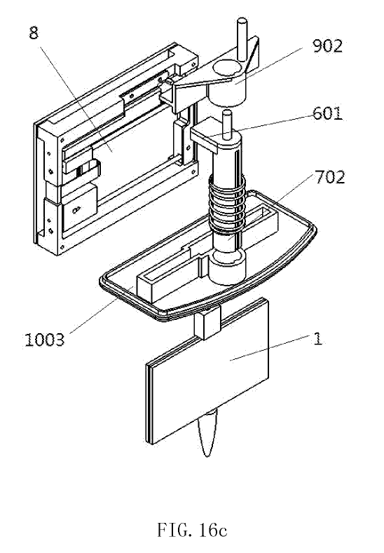

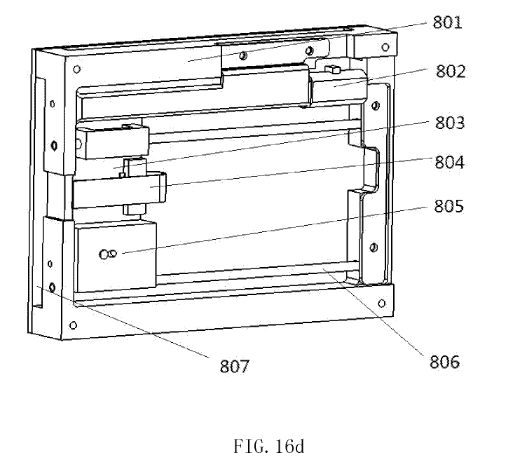

In the third better embodiment of the micro-fluidic chip in the urine analysis system, as shown in FIG. 12 (Three-dimensional model diagram), the micro-fluidic chip was made into cylindrical with 15 fluid channels, wherein the liquid inlet end 5 was a long, cylindrical catheter with internal circulation ports, wherein the liquid outlet end 4 was cylindrical with larger cross-sectional circulation ports inside. As shown in FIG. 13, the micro-fluidic chip was bonded with a basal layer 107, an interlayer 108 and an upper layer 109. The liquid inlet end 5 and the basal layer 107 were made into an assembly unit, the liquid outlet end 4 and the upper layer 109 were made into an assembly unit, and the basal layer 107 also had transparent circular sidewalls 110 that can surround the interlayer 108. The fluid channel 3 was made into the channel 307 on the lower surface of the interlayer 108, the channel 308 on the side surface and the channel 309 on the upper surface. The test strip 2 was located on the side surface of the interlayer 108 and in contact with channel 308. In service use, the urine sample entered micro-fluidic chip 1 from the liquid inlet end 5, it flew into the channel 308 through the channel 307 of the fluid channel 3, and then flew into channel 309, and flew out from the liquid outlet end 4. The test strip 2 was immersed in urine sample when urine sample pass through the channel 308, making the test strip 2 start chemical reaction and change color. In this embodiment, the test items of 15 test strips could be hematuria, leukocyte esterase, nitrite, urobilinogen, bilirubin, urine protein, microalbuminuria, glucose, ketone, PH, specific gravity, color. As for technician of this field, they can achieve the same goals using common means in their fields based on the basal layer 107, the interlayer 108 and the upper layer 109 of this embodiment. The common means were different forms of fluid channels, such as a simple replacement of the circular sidewalls 110 of the basal layer 107 to the upper layer 109, or making the part of circular sidewalls 110 into a single part, or fixing part of the fluid channels in the interlayer into the upper layer 109 and the basal layer 108, etc., these changes should be treated as equivalent as this embodiment.

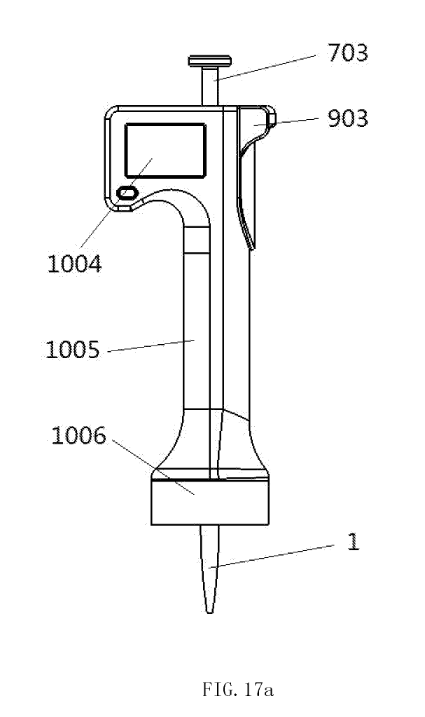

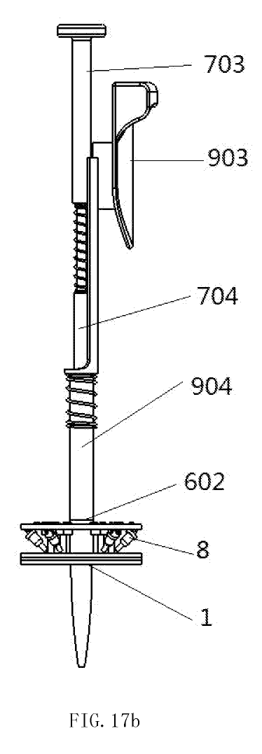

In the fourth better embodiment of the micro-fluidic chip in the urine analysis system, as shown in FIG. 14 (three-dimensional model diagram), the micro-fluidic chip was made into truncated cone-shaped with 15 fluid channels, wherein the liquid inlet end 5 was a long, cylindrical catheter with internal circulation ports, wherein the liquid outlet end 4 was cylindrical with larger cross-sectional circulation ports inside. As shown in FIG. 15, the micro-fluidic chip was bonded with a basal layer 111, an interlayer 112 and an upper layer 113. The liquid inlet end 5 and the basal layer 111 were made into an assembly unit, the liquid outlet end 4 and the upper layer 113 were made into an assembly unit, and the upper layer 113 also had windows 114 corresponding to each test strip 2. The fluid channel 3 was made into the channel 307 on the lower surface of the interlayer 112, the channel 308 on the side surface and the channel 309 on the upper surface. The test strip 2 was located on the side surface of the interlayer 112 and in contact with channel 308. In service use, the urine sample entered micro-fluidic chip 1 from the liquid inlet end 5, it flew into the channel 308 through the channel 307 of the fluid channel 3, and then flew into channel 309, and flew out from the liquid outlet end 4. The test strip 2 was immersed in urine sample when urine sample pass through the channel 308, making the test strip 2 start chemical reaction and change color. In this embodiment, the test items of 15 test strips could be hematuria, leukocyte esterase, nitrite, urobilinogen, bilirubin, urine protein, microalbuminuria, glucose, ketone, PH, specific gravity, color. As for technician of this field, they can achieve the same goals using common means in their fields based on the basal layer 111, the interlayer 112 and the upper layer 113 of this embodiment. The common means were different forms of fluid channels, such as replacing the windows on the upper layer 113 to transparent material, or replacement of the upper layer and the basal layer, etc., these changes should be treated as equivalent as this embodiment.

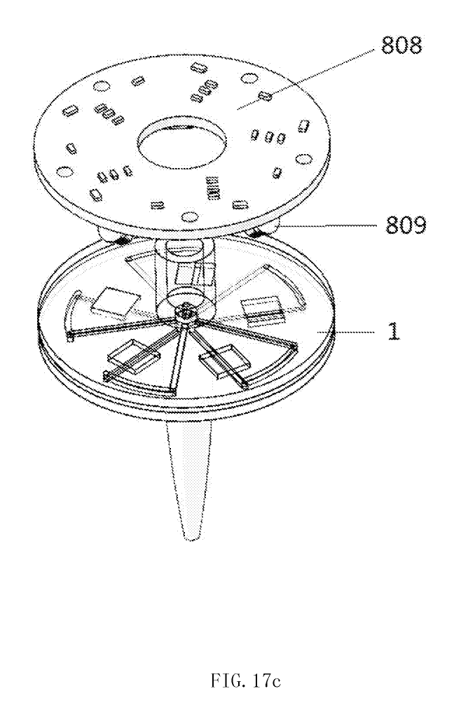

In the first better embodiment of the urine analyzer in the urine analysis system, as shown in FIGS. 16a, 16b and 16c of this embodiment, if we use rectangular micro-fluidic chip, and the shape of the urine analyzer was rectangular. The urine analyzer 10 in this embodiment contained the top and side shell 1001 and the bottom shell 1003. The top also had the display screen 1002, the sampling key 701 and the unloading key 901 which directly connected the unloading device below. The suction device 702 was made into a cylindrical structure, and the unloading device 902 was nested on the exterior of the cylindrical suction device 702 and connected with a spring device. The suction device 702 was connected with the connection port 601 through the internal pipe. In service use, The micro-fluidic chip 1 was inserted into the urine analyzer through the gap between the bottom shell 1003 at the bottom of the urine analyzer, so that the liquid outlet end 4 at the top of micro-fluidic chip 1 was connected with the junction 601. A detector 8 was layout in the flank of the micro-fluidic chip 1, and the main circuit board 807 was fixed at the hemi-frame 801 the linear motor with lead screw 802 with two sliding guide rods 806 which was arranged in parallel inside the frame 801, and the mobile circuit board 803 stretch across two sliding guide rods 806 which was connected with the linear motor with lead screw 802. The detection sensor 805 was arranged on mobile circuit boards 803, the mobile circuit board 803 connected with the main circuit board 807 through flexible flat cable 804. During sample testing, the linear motor with lead screw 802 was started to drive the motion of the mobile circuit board 803 through sliding guide rods 806. In this embodiment, test strip 2 in the rectangular micro-fluidic chip line up in order in the route of the detection sensor 805. When the detection sensor 805 moved, it could collect the color data of the test strip 2 in turn.

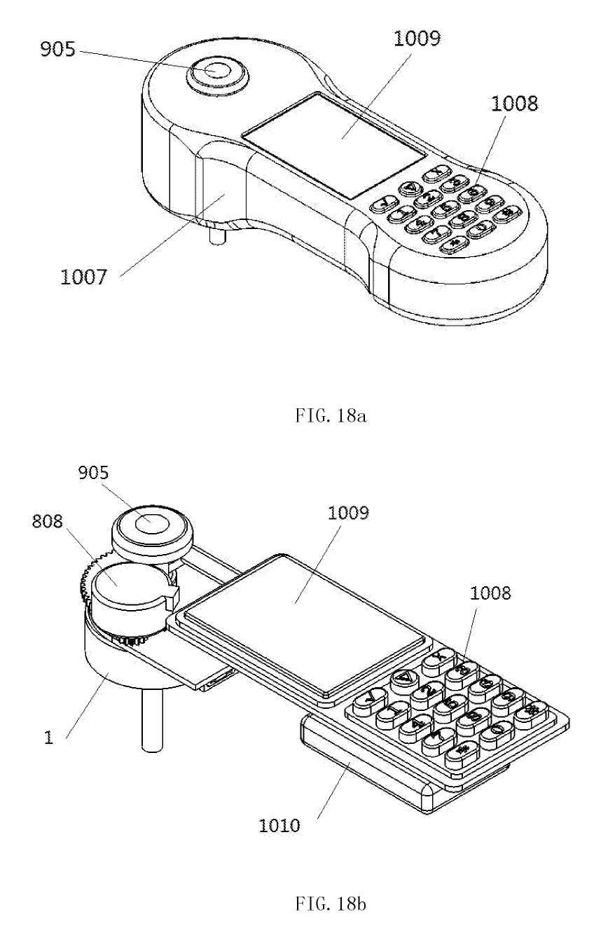

In the second better embodiment of the urine analyzer in the urine analysis system, as shown in FIGS. 17a, 17b and 17c of this embodiment, if the urine analyzer 10 was made into handheld pipettor, accordingly, discoid micro-fluidic chip should fixed under the urine analyzer 10 in the form of pipettor which was replaceable. The pipettor-shaped top shell 1005 was integrated with a main electrical component 1004 on the flank, the main electrical component 1004 included circuit board, keys, display screen, battery and wireless connection module. The bottom shell 1006 was provided with a detector 8 inside. The method of color data collecting was different with the first better embodiment, it was arranged several detection sensors corresponding to each test strip. There were sampling key 703 connected with the suction device 704 and the unloading key 903 connected with the unloading device 904. The unloading device was housed concentric to the exterior of the suction device. The lower part of the suction device 704 was connected with the micro-fluidic chip 1 as a connection port 602.

When using the urine analyzer of this embodiment for urine analysis, users can start the urine analyzer, then install the micro-fluidic chip into the bottom of the urine analyzer, and then holding the urine analyzer with single hand and put the liquid inlet end of micro-fluidic chip into the bottom of the urine analyzer. Then press the sampling key, so that urine sample will be sucked into the micro-fluidic chip, wait for a period of time, the reaction was completed and urine analyzer collects the color data of test strips. Finally, users could press the unloading key to separate the micro-fluidic chip used from the urine analyzer.

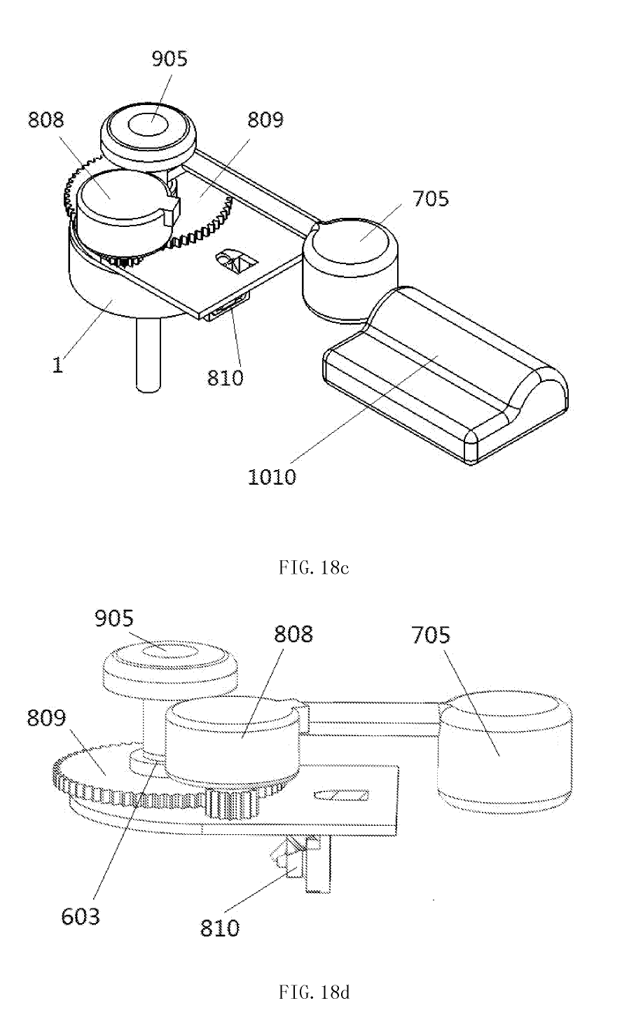

In the third better embodiment of the urine analyzer in the urine analysis system, as shown in FIGS. 18a, 18b and 18c of this embodiment. The micro-fluidic chip 1 was cylindrical, while the detector 8 in urine analyzer 10 is more complicated, there was a slewing mechanism which was used to drive the micro-fluidic chip 1 rotating and make the test strips move to the detection sensor 810. The urine analyzer of this embodiment included a shell 1007, a key and circuit board 1008, a display screen 1009, a printing mode 1010, the electric suction device 705, a connection port 603, an unloading device 905 and a special detector 8. This special detector 8 includes a detection sensor 810 which contains a sensor, an electrical machinery 808 used to drive the rotation of the micro-fluidic chip, a transmission gear 809.

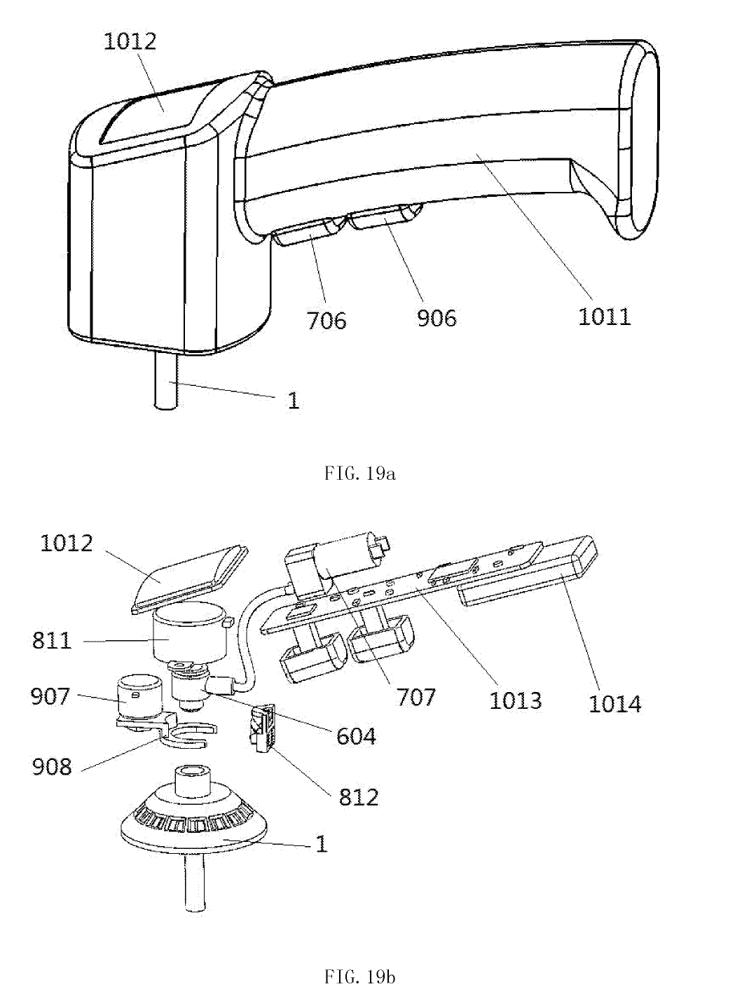

In the fourth better embodiment of the urine analyzer in the urine analysis system, the shape of urine analyzer is made into handheld electronic thermometer as shown in FIGS. 19a and 19b of this embodiment. The micro-fluidic chip is truncated cone-shaped, and the shape of urine analyzer was made into handheld electronic thermometer, there was a display screen 1012 at the top of urine analyzer while a sampling key 706 and an unloading key 906 at the front handle which lay circuit board 1013 and battery 1014. The connection of the urine analyzer connected with the micro-fluidic chip 1 was a connection port 604. It was connected with a rotary motor 811 and a pump 707 through a pipe, there was a detection sensor 812 located on the flank of the micro-fluidic chip. The lateral connection port 604 was equipped with an electromagnet 907 and a mechanical arm 908 which was used to unload the micro-fluidic chip 1. In this embodiment, the suction device 7 and the chip unloading device 9 were electrically controlled.

It is necessary to state that this case included but not limited to these three kinds of detecting test strips: Firstly, micro-fluidic chip and detector was relatively rest (namely, several sensors corresponds to each test strip), secondly, detector was stationary while micro-fluidic chip moves with detector in opposite direction, thirdly, micro-fluidic chip kept stationary and detector moves with detector in opposite direction. The sensors in the detector can move straight or rotate as well as the micro-fluidic chip. Although the micro-fluidic chips in the embodiments above are rotate, it could be move straight. These all protected by this case. For example, users could use the plan of first kind of detecting test strips if the test items are less, and adopt the second or third kind of detecting test strips according to the structure and appearance of the analyzer if there are many test items. The combined or replaced designs of the above schemes were the common means of the field.

The above embodiments of the urine analyzer of the invention, wherein the suction device, detector and unloading device have different forms, technicians of this fields can simply obtain a new technical proposal based on the replaced and combined designs of urine analyzer and micro-fluidic chips. For example, in the second better embodiment of handheld pipettor-shaped urine analyzer, using electric suction device, or electric unloading device, etc., these changes should be treated as equivalent as this embodiment.

Despite the implementing scheme of the invention is disclosed to the public, but it is not just limited to listed application mentioned above in the specification and embodiments, it can be suitable for various relevant fields. As for technicians in this field, it can be easily modified. Therefore, the invention is not limited to specific details and illustrations shown and described here within the claims.

1. A urine analysis system comprising a urine analyzer and a micro-fluidic chip, wherein
the micro-fluidic chip is provided with several test strips and fluid channels corresponding to each test strip; and
a liquid inlet end is arranged at a lower end of the micro-fluidic chip and connected to one end of the fluid channel;
a liquid outlet end is arranged at an upper end of the micro-fluidic chip and connected to the other end of the fluid channel;
the urine analyzer comprises a detector for detecting the test strips, a connection port for the micro-fluidic chip; and
a suction device connects the liquid outlet end of the micro-fluidic chip through the connection port.

2. The urine analysis system as claimed in claim 1, characterized in that test items of the urine analysis system comprises one or more of urobilinogen, bilirubin, ketone, nitrite, erythrocyte, leukocyte esterase, specific gravity, PH, protein, microalbumin(MALB), glucose, ascorbic acid, creatinine, calcium, and color.

3. The urine analysis system as claimed in claim 2, characterized in that N fluid channels and N test strips are arranged in the N fluid channels, each test strip corresponds to one fluid channel, and N is an integer not less than 1 and not greater than 15; the test strips react with the urine sample passing through the fluid channel and change color.

4. The urine analysis system as claimed in claim 3, characterized in that at least one of the two larger sides of the test strip area is encapsulated in transparency.

5. The urine analysis system as claimed in claim 4, characterized in that at least one of two larger sides of the test strip area is encapsulated in exposure.

6. The urine analysis system as claimed in claim 3, characterized in that the shape of micro-fluidic chip is rectangle, and the fluid channels are parallelly distributed on the micro-fluidic chip.

7. The urine analysis system as claimed in claim 3, characterized in that the shape of micro-fluidic chip is discoid, and the fluid channels are radially distributed on the micro-fluidic chip.

8. The urine analysis system as claimed in claim 3, characterized in that the shape of micro-fluidic chip is cylindrical, and the test strips are distributed on the cylindrical side.

9. The urine analysis system as claimed in claim 3, characterized in that the shape of micro-fluidic chip is a circular truncated cone, and the test strips are distributed on the truncated cone side.

10. The urine analysis system as claimed in claim 1, characterized in that the urine analyzer includes an unloading device for unloading the micro-fluidic chip.

11. The urine analysis system as claimed in claim 10, characterized in that the detector is arranged by the side of the micro-fluidic chip and includes a movable detection sensor.

12. The urine analysis system as claimed in claim 10, characterized in that the detector includes N detection sensors, and the number of detection sensors is the same as the number of test strips.

13. The urine analysis system as claimed in claim 10, characterized in that the detector includes a detection sensor and a mechanical device that moves the micro-fluidic chip; the detection sensor is used to collect the color data of the test strips.

14. The urine analysis system as claimed in claim 10, characterized in that the urine analyzer also includes a top shell, a side shell and a bottom shell, and the top of the urine analyzer is provided with a display screen, a sampling key and an unloading key; the unloading device is connected under the unloading key; the shape of suction device is cylindrical and the unloading device is nested outside the suction device; the suction device is connected with the connection port through an internal pipe.

15. The urine analysis system as claimed in claim 10, characterized in that the detector is located in flank of the micro-fluidic chip, the detector also includes a main circuit board which is fixed at a frame, a linear motor with a lead screw and two parallelly sliding guide rods that are arranged inside the frame, and a mobile circuit board stretch across the two sliding guide rods which is connected with the linear motor with lead screw; the detection sensor is arranged on the mobile circuit board, the mobile circuit board connects with the main circuit board through a flexible flat cable.

16. The urine analysis system as claimed in claim 10, characterized in that the urine analyzer is in the shape of a handheld pipettor, including a top shell, a bottom shell, and the flank of the top shell is integrated with a main electrical component, which includes circuit board, keys, a display screen, a battery and a wireless connection module; the top of the urine analyzer is also provided with a sampling key connecting the suction device and an unloading key connecting the unloading device; the unloading device is housed concentric to the exterior of the suction device; the lower part of the suction device is connected with the micro-fluidic chip as the connection port.

17. The urine analysis system as claimed in claim 10, characterized in that the detector includes a detection sensor which contains one sensor, an electrical motor used to drive the micro-fluidic chip rotating, transmission gear, and a slewing mechanism which is used to drive the micro-fluidic chip rotating and make the test strips move to the detection sensor.

18. The urine analysis system as claimed in claim 10, characterized in that the shell of the urine analyzer is handheld and has a display screen on the top, the shell is provided with a sampling key and an unloading key, and the shell is equipped with circuit boards and batteries inside; the urine analyzer connects the micro-fluidic chip through the connection port, and the lateral connection port is equipped with an electromagnet and an mechanical arm which is used to unload the micro-fluidic chip; the suction device and the chip unloading device are electrically controlled.

19. The urine analysis system as claimed in claim 10, characterized in that the detector includes a rotary motor connected with the connection port, a pump connected with the connection port through a pipe, and a detection sensor located on the flank of the micro-fluidic chip.

20. A method for urine analysis, characterized in that including the following steps:
Step 1) loading chip: inserting the micro-fluidic chip used for urine analysis into the urine analysis system so that the liquid outlet end of the fluid channel of the micro-fluidic chip is connected with the suction device in the urine analysis system, test strips which can react with the urine sample to be tested are placed in the micro-fluidic chip;
Step 2) sampling: inserting the liquid inlet end of the micro-fluidic chip into the urine sample to be tested, and start the suction device to let the urine sample flow into the micro-fluidic chip;
Step 3) reacting: after Step 2) and the urine sample to be tested being absorbed in the microfluidic chip, the test strips reacting with the urine sample and produce color changes;
Step 4) obtaining color data: the urine analysis system having a detector which obtains the color data of the test strips in the micro-fluidic chip which reacts with the urine sample to be tested;
Step 5) analyzing results: the urine analysis system analyzing the concentration of components in the urine sample to obtain the urine analysis results according to the color data of the test strips obtain in the Step 4); and
Step 6) unloading the chip: unloading the micro-fluidic chip from the urine analysis system.

21. The method for urine analysis as claimed in claim 20, characterized in that the test items of the method for urine analysis include but are not limited to one or more of urobilinogen, bilirubin, ketone, nitrite, erythrocyte, leukocyte esterase, specific gravity, PH, protein, microalbumin (MALB), glucose, ascorbic acid, creatinine, calcium, or color.

22. The method for urine analysis as claimed in claim 21, characterized in that N fluid channels and N test strips are arranged in the N fluid channels, each test strip corresponds to one fluid channel, and N is an integer not less than 1 and not greater than 15; the test strips react with the urine sample passing through the fluid channel and change color.

23. The method for urine analysis as claimed in claim 21, characterized in that the shape of micro-fluidic chip is rectangle, and the fluid channels are parallelly distributed on the micro-fluidic chip.

24. The method for urine analysis as claimed in claim 21, characterized in that the shape of micro-fluidic chip is discoid, and the fluid channels are radially distributed on the micro-fluidic chip.

25. The method for urine analysis as claimed in claim 21, characterized in that the shape of micro-fluidic chip is cylindrical, and the test strips are distributed on the cylindrical side.

26. The method for urine analysis as claimed in claim 21, characterized in that the shape of micro-fluidic chip is a circular truncated cone, and the test strips are distributed on the truncated cone side.

27. The method for urine analysis as claimed in claim 21, characterized in that the urine analyzer includes an unloading device for unloading the micro-fluidic chips.

28. The method for urine analysis as claimed in claim 21, characterized in that the detector is arranged by the side of the micro-fluidic chip and includes a movable detection sensor; the detection sensor moves to the flank of each test strip of the micro-fluidic chip to collect the color data of the test strips.

29. The method for urine analysis as claimed in claim 21, characterized in that the detector includes N detection sensors, and the number of detection sensors is the same as the number of test strips; the N detection sensors are respectively corresponding to the color data of the N test strips in the micro-fluidic chip.

30. The method for urine analysis as claimed in claim 21, characterized in that the detector includes a detection sensor and a mechanical device that moves the micro-fluidic chip; the mechanical device drives the micro-fluidic chip moving so that the test strips of the microfluidic chip entering the detection sensor by order; the detection sensor is used to collect the color data of the test strips.

31. The method for urine analysis as claimed in claim 21, characterized in that the urine analyzer also includes a top shell, a side shell and a bottom shell, and the top of the urine analyzer is provided with a display screen, a sampling key and an unloading key; the unloading device is connected under the unloading key; the shape of suction device is cylindrical and the unloading device is nested outside the suction device; the suction device is connected with the connection port through an internal pipe.

32. The method for urine analysis as claimed in claim 21, characterized in that the detector is located in flank of the micro-fluidic chip, the detector also includes: a main circuit board which is fixed at a frame, a linear motor with a lead screw and two parallelly sliding guide rods are arranged inside the frame, and a mobile circuit board stretch across the two sliding guide rods which is connected with the linear motor with the lead screw; the detection sensor is arranged on the mobile circuit board, the mobile circuit board connects with the main circuit board through flexible flat cable.

33. The method for urine analysis as claimed in claim 21, characterized in that the urine analyzer is in the shape of a handheld pipettor, including a top shell, a bottom shell, and the flank of the top shell is integrated with a main electrical component, which includes a circuit board, keys, a display screen, a battery and a wireless connection module; the top of the urine analyzer is also provided with a sampling key connecting the suction device and an unloading key connecting the unloading device; the unloading device is housed concentric to the exterior of the suction device; the lower part of the suction device is connected with the micro-fluidic chip as the connection port.

34. The method for urine analysis as claimed in claim 21, characterized in that the detector includes a detection sensor which contains one sensor, an electrical motor used to drive the micro-fluidic chip rotating, a transmission gear, and a slewing mechanism which is used to drive the micro-fluidic chip rotating and make the test strips move to the detection sensor.

35. The method for urine analysis as claimed in claim 21, characterized in that the shell of the urine analyzer is handheld and has a display screen on the top, the shell is provided with a sampling key and an unloading key, and the shell is equipped with circuit boards and batteries inside; the analyzer connects the micro-fluidic chip through the connection port, and the lateral connection port is equipped with an electromagnet and an mechanical arm which is used to unload the micro-fluidic chip; the suction device and the chip unloading device are electrically controlled.

36. The method for urine analysis as claimed in claim 21, characterized in that the detector includes a rotary motor connected with the connection port, a pump connected with the connection port through a pipe, and a detection sensor located on the flank of the micro-fluidic chip.

37. The method for urine analysis as claimed in claim 21, characterized in that the part of the micro-fluidic chip towards the detector is encapsulated in exposure or in transparency.
